(19) United States
(12) Patent Application Publication
(10) Pub. No.: US 2012/0287750 A1
(43) Pub. Date: Nov. 15, 2012

(54) IMAGING APPARATUS

(75) Inventors: Szabolcs Deladi, Eindhoven (NL); Nenad Mihajlovic, Eindhoven (NL); Drazenko Babic, Eindhoven (NL); Jan Suijver, Eindhoven (NL); Yan Shi, White Plains, NY (US)

(73) Assignee: KONINKLIJKE PHILIPS ELECTRONICS N.V., EINDHOVEN (NL)

(21) Appl. No.: 13/522,789

(22) PCT Filed: Jan. 12, 2011
(86) PCT No.: PCT/IB11/50129

§ 371 Date: Jul. 18, 2012

Related U.S. Application Data
(60) Provisional application No. 61/296,053, filed on Jan. 19, 2010.

(51) Int. Cl. G01S 15/89; A61B 8/12

(52) U.S. Cl. 367/7

(57) ABSTRACT

The invention relates to an imaging apparatus (1) for imaging an interior of an object (2). The imaging apparatus (1) comprises a first ultrasound sensor and a second ultrasound sensor for sensing the interior of the object at different frequencies, wherein the ultrasound sensing signals from the first ultrasound sensor are used for generating a first ultrasound image and a the ultrasound sensing signals from the second ultrasound sensor are used for generating a second ultrasound image. A larger frequency generally provides a smaller depth of penetrating the interior of the object and a larger spatial resolution than a smaller frequency. The imaging apparatus (1) can therefore provide the capability of simultaneously imaging the interior of the object with different spatial resolutions and at different penetration depths. This allows the imaging apparatus to improve the quality of imaging the interior of the object.

### FIELD OF THE INVENTION

The invention relates to an imaging apparatus, imaging method and imaging computer program for imaging an interior of an object. The invention relates further to an influencing apparatus, an influencing method and an influencing computer program for influencing an interior of an object.

### BACKGROUND OF THE INVENTION

U.S. Pat. No. 7,396,332 B2 discloses a single transducer element that is capable of oscillation at a plurality of natural resonant frequencies which may be used in an ultrasonic imaging catheter assembly including a catheter body configured to be inserted and guided through the vascular system of a living being. The ultrasonic imaging catheter assembly comprises a lumen and a rotatable imaging core adapted to pass through the lumen, wherein the imaging core includes a flexible drive shaft. Since the transducer element is capable of oscillation at a plurality of natural resonant frequencies, a user can switch from one frequency to another in order to improve the depth of field or resolution without having to switch out the catheter or imaging core.

### SUMMARY OF THE INVENTION

It is an object of the present invention to provide an imaging apparatus, imaging method and imaging computer program for imaging an interior of an object, wherein the quality of imaging the interior of the object can be improved. It is a further object of the present invention to provide an influencing apparatus, an influencing method and an influencing computer program for influencing an interior of an object, which use the improved imaging.

In a first aspect of the present invention an imaging apparatus for imaging an interior of an object is presented, wherein the imaging apparatus comprises:
a first image generation device including:
a) a first ultrasound sensor for sensing the interior of the object at a first frequency, wherein first ultrasound sensing signals are generated being indicative of the interior of the object,
b) a second ultrasound sensor for sensing the interior of the object at a second frequency, wherein second ultrasound sensing signals are generated being indicative of the interior of the object, wherein the first frequency is smaller than the second frequency such that the first ultrasound sensor is adapted to sense the interior of the object with smaller spatial resolution and the second ultrasound sensor is adapted to sense the interior of the object with larger spatial resolution,
c) an ultrasound image generation unit for generating a first ultrasound image having a smaller spatial resolution from the first ultrasound sensing signals and a second ultrasound image having a larger spatial resolution from the second ultrasound sensing signals,
a housing for housing at least the first ultrasound sensor and the second ultrasound sensor, the housing being adapted to be introducible into the object.

Since a first ultrasound sensor and a second ultrasound sensor are used for sensing the interior of the object at different frequencies, wherein the ultrasound sensing signals from these ultrasound sensors are used for generating the first ultrasound image and the second ultrasound image, the interior of the object can be sensed simultaneously with different spatial resolutions. Moreover, a larger frequency has generally a smaller depth of penetrating the interior of the object than a smaller frequency. Thus, at least two images can simultaneously be generated, which image the interior of the object at different depths. The imaging apparatus can therefore provide the capability of simultaneously imaging the interior of the object with different spatial resolutions and at different penetration depths. This allows the imaging apparatus to improve the quality of imaging the interior of the object.

The housing is preferentially a catheter or an interventional needle.

The imaging apparatus can comprises further elements being incorporated in the housing. For example, further ultrasound sensors, for example, phased ultrasound arrays, other sensing elements like electrical or optical sensing elements, biopsy elements for performing a biopsy operation, energy application elements like ablation elements, for example, ablation electrodes, ablation cryo elements, ablation optical elements, et cetera.

The first and second ultrasound sensors can be adapted to sense the interior of the object in different directions. Moreover, the first image generation device can comprise at least two pairs of the first ultrasound sensor and the second ultrasound sensor, wherein different pairs sense the interior of the object in different directions.

If the imaging apparatus comprises further ultrasound sensors, these further ultrasound sensors have preferentially frequencies being different to the first frequency and the second frequency, in order to sense the interior of the object with a different spatial resolution and at a different depths.

The first frequency and the second frequency are preferentially center frequencies, i.e. as it is well known to a person skilled in the art an ultrasound sensor does not operate at a single frequency but at a frequency range centered around a center frequency. Thus, an ultrasound sensor generally comprises a bandwidth having a center frequency. The first and second frequencies are preferentially the center frequencies of the bandwidths of the first and second ultrasound sensors, respectively.

It is preferred that the imaging apparatus further comprises a second image generation device for generating a third image of the object and an overlay unit for overlaying the third image with at least one of the first ultrasound image and the second ultrasound image.

It is further preferred that the second image generation device comprises:
a) a radiation source for generating radiation for traversing the object,
b) a detector for generating detection values depending on the radiation after having traversed the object,
c) an image reconstruction unit for reconstructing the third image from the generated detection values.

The radiation source is preferentially an X-ray source and the detector is preferentially an X-ray detector.

The second image generation device can also be another imaging modality. For example, the second image generation device can be a magnetic resonance imaging modality or a nuclear imaging modality like a positron emission tomography modality or a single photon emission computed tomography modality.

The image reconstruction unit is preferentially adapted to reconstruct a projection image of the object from the generated detection values, wherein the image reconstruction unit can be adapted to arrange the generated detection values side-by-side for reconstructing the projection image. However, the image reconstruction unit can also be adapted to reconstruct, for example, a computed tomography image of the object, wherein the radiation source is adapted such that the generated radiation traverses the object in different directions, the detector is adapted to generated detection values depending on the radiation after having traversed the object in different directions, and the image reconstruction unit is adapted to reconstruct a computed tomography image from the generated detection values.

The third image is preferentially generated by an imaging modality not being an ultrasound imaging modality. The third image shows therefore features of the interior of the object, which may not or differently be shown on the first and second ultrasound images. The overlay image being an overlay of the third image and of at least one of the first ultrasound image and the second ultrasound image contains therefore more information regarding the interior of the object and can therefore improve the imaging of the object. For example, the first ultrasound image may comprise a smaller spatial resolution at a larger penetration depth, the second ultrasound image may comprise a larger spatial resolution at a smaller penetration depth, and the third image may comprise the lowest spatial resolution and may be a projection image, in particular, if the radiation source of the second image generation device is an X-ray source.

It is further preferred that the imaging apparatus comprises a registration unit for registering at least one of the first ultrasound image and the second ultrasound image with the third image. If the third image and the at least one of the first ultrasound image and the second ultrasound image are registered with respect to each other, they can be more correctly overlaid by the overlay unit, thereby further improving the quality of imaging the interior of the object. The registration and the generation of the overlay image are preferentially performed in realtime.

Preferentially, an element having a known spatial relationship to the first ultrasound sensor and the second ultrasound sensor and being visible in the third image is used by the registration unit for registering the images. This element is, for example, the first ultrasound sensor itself, the second ultrasound sensor itself, a biopsy needle, et cetera.

The object can be a technical object like a machine, a pipeline or any other technical object of which the interior has to be imaged. The object can also be a person or an animal, wherein the inside of the person or the animal, in particular, the inside of an organ, vessel, et cetera, has to be imaged.

It is further preferred that the imaging apparatus comprises optical fibers for illuminating the interior of the object with light and for receiving the light from the interior of the object and a spectrometer for spectrally investigating the received light, wherein the optical fibers are housed within the housing. The imaging apparatus preferentially comprises one or several optical fibers for illuminating the interior of the object and one or several optical fibers for receiving light from the interior of the object, which has preferentially been scattered by the interior of the object. The spectrometer generates a spectrum of the received light. The spectrometer can further be adapted to determine information about the interior of the object from the generated spectrum. For example, for certain materials like fat, water, blood, oxygen, et cetera spectra can be determined by calibration, wherein these spectra can be stored in a storing unit of the spectrometer. After a spectrum has been generated for an actual measurement, this spectrum can be compared with the spectra stored in the storing unit for determining the material actually illuminated by the light. In particular, if it is assumed that different tissue types consist of different material combinations, the spectrometer can be adapted to determine the respective tissue type by investigating the spectrum. For example, if it is known that a certain tissue type consists of a certain material combination, by spectrally analyzing the actually measured spectrum it can be determined which material combination is actually illuminated and, thus, which tissue type is actually illuminated. The imaging apparatus can therefore be adapted to allow determining which tissue type is in front of a catheter tip or a needle tip.

It is further preferred that the imaging apparatus comprises a driving unit for driving the first ultrasound sensor and the second ultrasound sensor. The first ultrasound sensor and the second ultrasound sensor are preferentially driven by the same driving unit. Since it is not necessary that each ultrasound sensor is connected to its own driving unit, the wiring within the housing for connecting the ultrasound sensors with the driving unit can be simplified and requires less space within the housing. This allows using a housing having a smaller diameter and/or integrating further elements within the housing.

It is further preferred that the driving unit is connected to the first ultrasound sensor and the second ultrasound sensor via a single wire. This can further simplify the wiring for connecting the ultrasound sensors to the driving unit and can reduce the space needed for the wiring within the housing.

It is further preferred that the driving unit is adapted to receive combined ultrasound sensing signals from the first ultrasound sensor and the second ultrasound sensor comprising the first ultrasound sensing signals and the second ultrasound sensing signals, wherein the driving unit comprises a filtering unit for filtering the first ultrasound sensing signals and the second ultrasound sensing signals out of the combined ultrasound sensing signals.

For filtering the first ultrasound sensing signals the filtering unit is preferentially adapted to Fourier transform the combined ultrasound sensing signals, to use a first frequency bandpass filter filtering a bandpass comprising the first frequency, and to inversely Fourier transform the bandpass filtered combined ultrasound signals resulting in the first ultrasound sensing signals. For filtering the second ultrasound sensing signals the filtering unit is preferentially adapted to Fourier transform the combined ultrasound sensing signals, to use a second frequency bandpass filter filtering a bandpass comprising the second frequency, and to inversely Fourier transform the bandpass filtered combined ultrasound signals resulting in the second ultrasound sensing signals.

The first frequency bandpass filter can, for example, be determined by performing a calibration measurement, wherein the bandwidth and the center frequency of the first ultrasound sensing signals are determined, when only first ultrasound sensing signals are present. Similarly, the second frequency bandpass filter can, for example, be determined by performing a calibration measurement, wherein the bandwidth and the center frequency of the second ultrasound sensing signals are determined, when only second ultrasound sensing signals are present. The determined bandwidths and center frequencies of the first and second ultrasound sensing signals define the first and second frequency bandpass filters, respectively.

Preferentially, the calibration measurements are performed only once or after a number of measurements have been performed, but not before each measurement. In particular, the manufacturer of the imaging apparatus can already perform the calibration measurements and store the determined first and second frequency bandpass filters in the filtering unit, or provide the determined first and second frequency bandpass filters to a user, which enters the first and second frequency bandpass filters into the filtering unit via a provided input unit like a keyboard, a mouse, or the like.

It is further preferred that the imaging apparatus comprises a navigation unit for allowing the housing to be navigated to a desired location within the object depending on at least the first ultrasound image and the second ultrasound image. Since the first ultrasound image and the second ultrasound image provide an improved imaging of the interior of the object, in particular, because different spatial resolutions and different imaging ranges are provided, a navigation based on this imaging can also be improved.

The navigation unit is preferentially adapted to allow the tip of the housing, in particular, of the catheter, to be navigated to the desired location within the object.

The navigation unit can also be adapted to allow the housing to be navigated depending on further images, in particular, depending on an overlay of at least one of the first ultrasound image and the second ultrasound image and the third image.

The navigation unit can be adapted to allow a user to navigate the housing completely by hand or semi-automatically depending on at least the first ultrasound image and the second ultrasound image. The navigation unit can also be adapted to navigate the housing fully automatically depending on at least the first ultrasound image and the second ultrasound image.

The first ultrasound image having a smaller spatial resolution and larger imaging range can be used to coarsely navigate the housing to the desired location, and the second ultrasound image having a finer spatial resolution and a smaller imaging range can be used to navigate the housing more precisely to or adjust the position of the housing precisely at the desired location.

The first ultrasound image can be used to image a first region of interest being located at a larger depth within object and the second ultrasound image can be used to image a second region of interest being located at a smaller depth within the object.

The first ultrasound sensor and the second ultrasound sensor are preferentially operated simultaneously, in order to provide feedback for coarse guidance and simultaneously high resolution information, in particular, from the vicinity of the tip of the housing, for example, from the tip of a biopsy needle. However, the first ultrasound sensor and the second ultrasound sensor can also be operated sequentially, in order to firstly provide the coarse information and subsequently provide the fine information.

It is further preferred that the first frequency and the second frequency are separated by at least the sum of half the bandwidth of the first ultrasound sensor and the second ultrasound sensor. It is also preferred that the first frequency is in the range of 1 to 10 MHz and the second frequency is in the range of 20 to 40 MHz. If the first frequency and the second frequency are within these ranges, they can easily be separated from each other by the filtering unit. Moreover, a first frequency within the above mentioned frequency range has a penetration depth within a body of a human being of up to 10 to 15 cm, which is very well suited for coarse navigation purposes, and a second frequency in the respective above mentioned frequency range allows to generate a second ultrasound image having a high spatial resolution, which is also well suited for navigation purposes.

Half the bandwidth is preferentially the half width at half maximum.

In a further aspect of the present invention an influencing apparatus for influencing an interior of an object is presented, wherein the influencing apparatus comprises:
an influencing element for influencing the object,
an imaging apparatus for generating a first ultrasound image and a second ultrasound image as defined in claim 1,
a navigation unit for navigating the influencing element to a desired location within the interior of the object depending on at least the first ultrasound image and the second ultrasound image.

The influencing element is preferentially a biopsy needle or an ablation element located at a tip of a catheter or interventional needle being the above mentioned housing. Thus, the influencing element is preferentially integrated into the catheter or interventional needle of the imaging apparatus. The navigation unit is preferentially adapted to navigate the biopsy needle or the ablation element to a desired location within the interior of the object depending on at least the first ultrasound image and the second ultrasound image.

In a further aspect of the present invention an imaging method for imaging an interior of an object is presented, wherein the imaging method comprises:
a) sensing the interior of the object at a first frequency by a first ultrasound sensor, wherein first ultrasound sensing signals are generated being indicative of the interior of the object,
b) sensing the interior of the object at a second frequency by a second ultrasound sensor, wherein second ultrasound sensing signals are generated being indicative of the interior of the object, wherein the first frequency is smaller than the second frequency such that the first ultrasound sensor senses the interior of the object with smaller spatial resolution and the second ultrasound sensor senses the interior of the object with larger spatial resolution,
c) generating a first ultrasound image having a smaller spatial resolution from the first ultrasound sensing signals and a second ultrasound image having a larger spatial resolution from the second ultrasound sensing signals by an ultrasound image generation unit,

wherein at least the first ultrasound sensor and the second ultrasound sensor are housed within a housing which is adapted to be introducible into the object.

In a further aspect of the present invention an influencing method for influencing an interior of an object is presented, wherein the influencing method comprises:
generating a first ultrasound image and a second ultrasound image by an imaging apparatus as defined in claim 12,
navigating an influencing element for influencing the object to a desired location within the interior of the object depending on at least the first ultrasound image and the second ultrasound image by a navigation unit, and
influencing the object with the influencing element.

In a further aspect of the present invention an imaging computer program for imaging an interior of an object is presented, wherein the imaging computer program comprises program code means for causing a computer to carry out the steps of the imaging method as defined in claim 12, when the computer program is run on a computer controlling an imaging apparatus as defined in claim 1.

In a further aspect of the present invention an influencing computer program for influencing an interior of an object is presented, wherein the influencing computer program comprises program code means for causing a computer to carry out the steps of the influencing method as defined in claim 13, when the computer program is run on a computer controlling an influencing apparatus as defined in claim 11.

It shall be understood that the imaging apparatus of claim 1, the influencing apparatus of claim 11, the imaging method of claim 12, the influencing method of claim 13, the imaging computer program of claim 14 and the influencing computer program of claim 15 have similar and/or identical preferred embodiments, in particular, as defined in the dependent claims.

It shall be understood that a preferred embodiment of the invention can also be any combination of the dependent claims with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of an imaging apparatus for imaging the interior of an object,

FIGS. 2 and 3 show schematically and exemplarily embodiments of distal ends of a catheter,

FIG. 4 shows exemplarily a combined ultrasound sensing signal,

FIG. 5 shows exemplarily a second ultrasound sensing signal,

FIG. 6 shows exemplarily a first ultrasound sensing signal,

FIGS. 7 to 9 show schematically and exemplarily further embodiments of distal ends of a catheter,

FIG. 10 illustrates an operation of several ultrasound sensors while navigating a distal end of a catheter to a target object,

FIG. 11 shows a flowchart exemplarily illustrating an embodiment of an imaging method for imaging an interior of an object, and

FIG. 12 shows a flowchart exemplarily illustrating an embodiment of an influencing method for influencing an interior of an object.

### DETAILED DESCRIPTION OF EMBODIMENTS

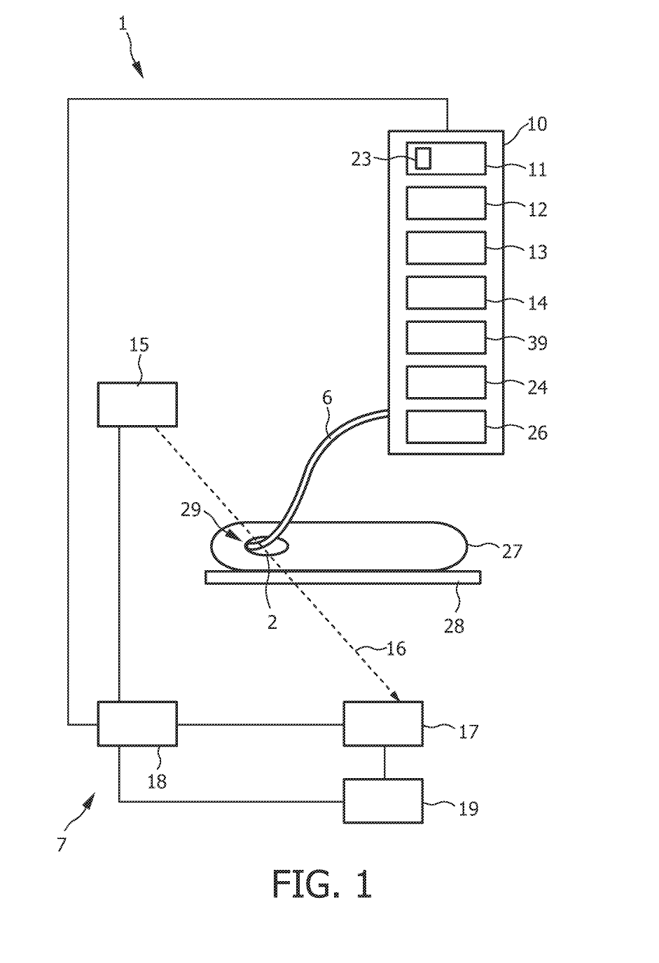

FIG. 1 shows schematically exemplarily an imaging apparatus for imaging an interior of an object 2. The object 2 is, in this embodiment, an inner organ 2 of a patient 27 who is located on a patient table 28. The apparatus 1 comprises a housing 6 for housing at least a first ultrasound sensor and a second ultrasound sensor, wherein the housing 6 is adapted to be introducible into the object 2. In this embodiment, the housing 6 is a catheter. The first ultrasound sensor and the second ultrasound sensor are preferentially located at the distal end 29 of the catheter 6.

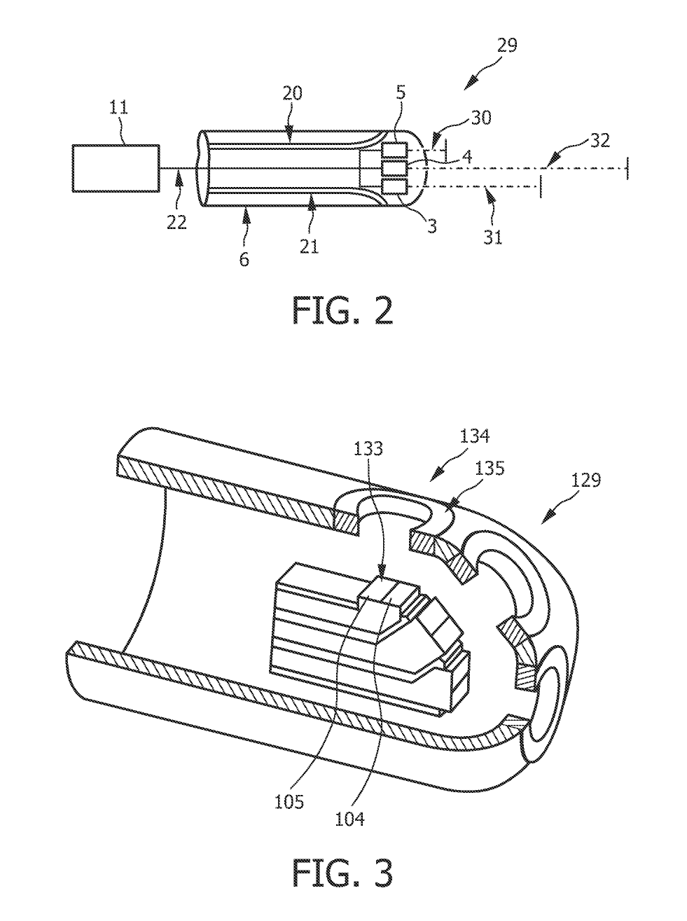

An embodiment of the distal end 29 of the catheter 6 is schematically and exemplarily shown in more detail in FIG. 2. The distal end 29 of the catheter 6 comprises a first ultrasound sensor 4 for sensing the interior of the object 2 at a first frequency, wherein first ultrasound sensing signals are generated being indicative of the interior of the object 2. The second ultrasound sensor 5 is adapted to sense the interior of the object 2 at a second frequency, wherein second ultrasound sending signals are generated being indicative of the interior of the object 2. The first frequency is smaller than the second frequency such that the first ultrasound sensor 4 senses the interior of the object with a smaller spatial resolution and the second ultrasound sensor 5 senses interior of the object 2 with a larger spatial resolution. The catheter 6 further comprises a third ultrasound sensor 3 for sensing the interior of the object 2 at a third frequency, wherein the third frequency is larger than the first frequency and smaller than the second frequency such that the third ultrasound sensor can sense the interior of the object with a spatial resolution being larger than the spatial resolution of the first ultrasound sensing signal and being smaller than the spatial frequency of the second ultrasound sensing signals.

Because of the different frequencies the imaging range 30 of the second ultrasound sensor 5 is smaller than the imaging range 31 of the third ultrasound sensor 3, and the imaging range 32 of the first ultrasound sensor 4 is larger than the imaging range 31 of the third ultrasound sensor 3. The different ultrasound sensing signals of the different ultrasound sensors 3, 4, 5 can therefore sense the interior of the object 2 in different penetration depths.

In FIG. 2 the ultrasound sensors 3, 4, 5 sense the interior of the object in the same direction. However, the ultrasound sensors can also be arranged to sense the interior of the object in different directions. Moreover, in a further embodiment of the distal end of the catheter the distal end can comprise at least two pairs of the first ultrasound sensor having a smaller frequency and the second ultrasound sensor having a larger frequency. Such an arrangement of these pairs within the distal end of a catheter is schematically and exemplarily shown in FIG. 3. In FIG. 3, the distal end 129 of the catheter comprises three pairs 133 of a first ultrasound sensor 104 and a second ultrasound sensor 105. The first ultrasound sensor 104 senses the interior of the object with a frequency being smaller than the frequency of the second ultrasound sensor 105. The three pairs 133 of ultrasound sensors are adapted to sense the interior of the object in different directions.

The distal end 129 of the catheter comprises three openings 134 which are surrounded by energy application elements 135 being preferentially ring electrodes. The openings 134 with the ring electrodes 135 are arranged such that the ultrasound waves can travel through the ring electrodes 135. The distal end of the catheter 129 shown in FIG. 3 comprises of course further elements which are not shown in FIG. 3 for clarity reason. For example, the distal end 129 of the catheter comprises wiring for connecting the ring electrodes 135 and the ultrasound sensors 104, 105 with respective control units outside the catheter. Moreover, the distal end 129 of the catheter can comprise further energy application elements, further sensing elements and/or biopsy elements. The ring electrodes 135 are preferentially used in an ablation procedure, in particular, in a radio frequency ablation procedure.

In this embodiment the openings 134 are not covered by window. However, in another embodiment the openings can be closed by a ultrasound transparent window like a polymethylpentene window. The contact between the ultrasound sensors and the interior of the object, in particular, of tissue of a human being, is mediated by an acoustically transparent matter like polymethylpentene or a physiological solution, for example, a saline solution.

Referring again to FIG. 1, the imaging apparatus 1 comprises a catheter control unit 10 including a driving unit 11 for driving the ultrasound sensors 3, 4, 5. The driving unit 11 is a single driving unit, i.e. the ultrasound sensors 3, 4, 5 are driven by the same driving unit. The connection of the ultrasound sensors 3, 4, 5 to the driving unit 11 is schematically indicated in FIG. 2. As can be seen in FIG. 2, the ultrasound sensors 3, 4, 5 are connected to the driving unit 11 via a single coaxial wire 22.

The driving unit 11 is adapted to receive combined ultrasound sensing signals from the different ultrasound sensors 3, 4, 5 comprising the first ultrasound sensing signals generated by the first ultrasound sensor 4, the second ultrasound sensing signals generated by the second ultrasound sensor 5, and the third ultrasound sensing signals generated by the third ultrasound sensor 3. The driving unit 11 comprises a filtering unit 23 for filtering the first ultrasound sensing signals, the second ultrasound sensing signals and the third ultrasound sensing signals out of the received combined ultrasound sensing signals.

For filtering, for example, the first ultrasound sensing signals, the filtering unit 23 is adapted to Fourier transform the combined ultrasound sensing signals, to use a first frequency bandpass filter filtering a bandpass comprising the first frequency, and to inversely Fourier transform the bandpass-filtered combined ultrasound signals resulting in the first ultrasound sensing signals. The second ultrasound sensing signals and the third ultrasound sensing signals can be filtered out of the combined ultrasound sensing signals accordingly.

Each ultrasound sensor operates with a certain bandwidth. Thus, the first ultrasound sensor operates at a first bandwidth, the second ultrasound sensor operates at a second bandwidth, and the third ultrasound sensor operates at a third bandwidth. The first frequency is the center frequency of the first bandwidth, the second frequency is the center frequency of the second bandwidth, and the third frequency is the center frequency of the third bandwidth. The first frequency bandpass filter is preferentially adapted such that it corresponds to the first bandwidth, a second frequency bandpass filter is preferentially adapted such that it corresponds to the second bandwidth, and the third frequency bandpass filter is preferentially adapted such that it corresponds to the third bandwidth.

The frequency bandpass filters can be determined by performing calibration measurements. For example, for determining the first frequency bandpass filter the first bandwidth and the first center frequency of the first ultrasound sensing signals can be determined, when only first ultrasound sensing signals are present, wherein this determined first bandwidth and first center frequency defines the first frequency bandpass filter. The second frequency bandpass filter and the third frequency bandpass filter can be determined in a similar way.

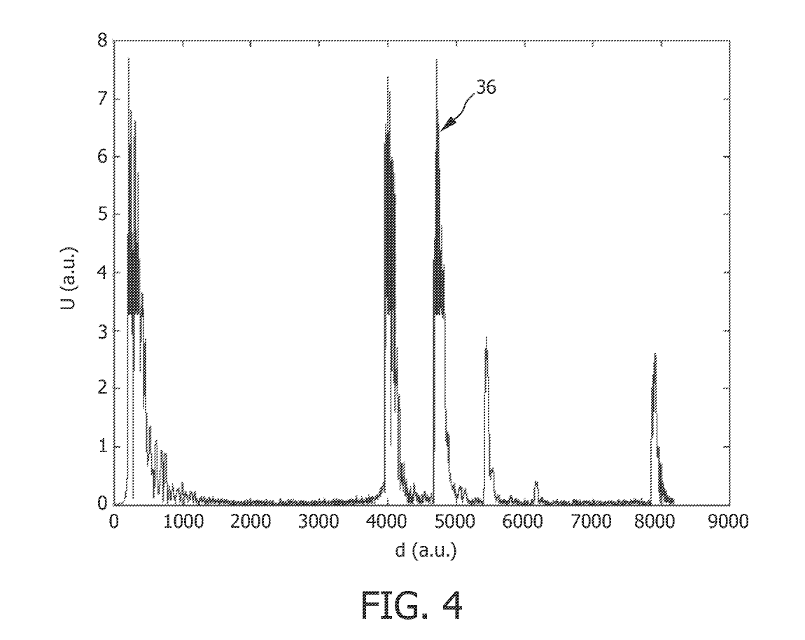

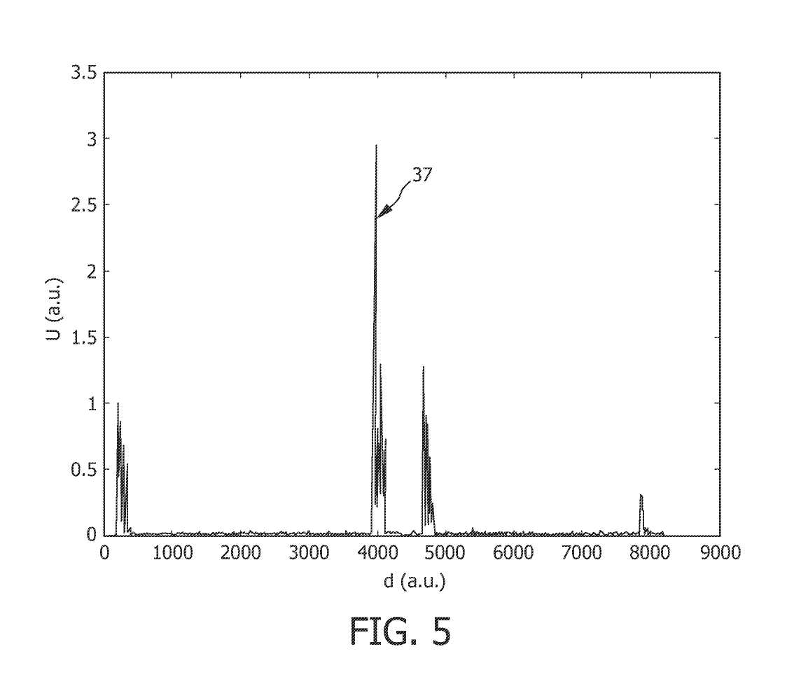

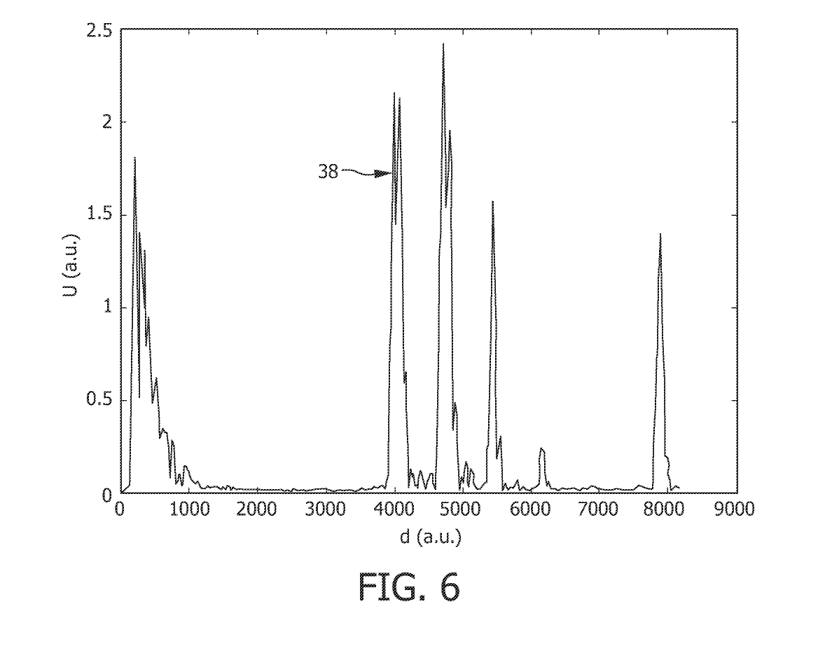

FIG. 4 shows exemplarily a combined ultrasound sensing signal 36 being a combination of a first ultrasound sensing signal and a second ultrasound sensing signal of the first and second ultrasound sensors 4, 5, respectively. FIG. 5 shows exemplarily the second ultrasound sensing signal after having filtered out of the combined ultrasound sensing signal 36 and FIG. 6 shows exemplarily the first ultrasound sensing signal 38 after having filtered out of the combined ultrasound sensing signal 36. In this embodiment, the ultrasound sensors preferentially comprise piezoelectric transducers generating electrical signals, if they receive ultrasound waves which have been reflected at different distances from the respective ultrasound sensor. In FIGS. 4 to 6 the vertical axis exemplarily denotes the voltage U of the electrical signal generated by the respective piezoelectric transducer in arbitrary units and the horizontal axis denotes the distance between the respective piezoelectrical transducer and the position at which the respective ultrasound wave has been reflected also in arbitrary units.

The catheter control unit 10 further comprises an ultrasound image generation unit 12 for generating a first ultrasound image having a smaller spatial resolution from the first ultrasound sensing signals and a second ultrasound image having a larger spatial resolution from the second ultrasound sensing signals. In this embodiment, the ultrasound image generation unit 12 is further adapted to generate a further ultrasound image having a spatial resolution being larger than the spatial resolution of the first ultrasound image and being smaller than the spatial resolution of the second ultrasound image from the third ultrasound sensing signals.

The driving unit 11 is adapted to drive the ultrasound sensors and to receive the ultrasound signals from the ultrasound sensors. The received ultrasound signals are provided to the image generation unit for generating, for example, B-mode ultrasound images if phased arrays are used as ultrasound sensors, and to generate A-mode and/or M-mode ultrasound images, if single transducers are used as ultrasound sensors.

The ultrasound sensors 3, 4, 5 form together with the driving unit 11 and the ultrasound image generation unit 12 a first image generation device.

The imaging apparatus 1 further comprises a second image generation device 7 for generating a further image of the object. In this embodiment, the second image generation device is a fluoroscopy device generating fluoroscopy images. The second image generation device 7 comprises a radiation source 15 for generating radiation 16 for traversing the object 2, a detector 17 for generating detection values depending on the radiation after having traversed the object 2, and an image reconstruction unit 19 for reconstructing the fluoroscopy image from the generated detection values. The radiation source 15 is an X-ray source and the detector 17 is an X-ray detector. The image reconstruction unit 19 arranges the generated detection values side-by-side for reconstructing a projection image being the fluoroscopy image.

In other embodiments, the second image generation device can also be another imaging modality. For example, projections can be generated in different directions and the image reconstruction unit can be adapted to reconstruct a computed tomography image of the object. Or the second image generation device can be a magnetic resonance imaging modality or a nuclear imaging modality like a positron emission tomography modality or a single photon emission computed tomography modality.

The radiation source 15, the detector 17 and the image reconstruction unit 19 are controlled by a fluoroscopy control unit 18 which preferentially also comprises a display for showing the fluoroscopy image.

As already mentioned above, the imaging apparatus, in particular, the catheter or interventional needle, can comprise further sensing elements. For example, as schematically shown in FIG. 2, the distal end 29 of the catheter 6 can comprise optical fibers 20, 21 for illuminating the interior of the object with light and for receiving light from the interior of the object 2. The optical fibers 20, 21 are connected to a spectrometer 39 in the catheter control unit 10 for spectrally investigating the received optical signals. The imaging apparatus preferentially comprises one or several optical fibers for illuminating the interior of the object and one or several optical fibers for receiving light from the interior of the object, which has preferentially been scattered by the interior of the object. The spectrometer 39 generates a spectrum of the received light. The spectrometer 39 can further be adapted to determine information about the interior of the object from the generated spectrum. For example, for certain materials like fat, water, blood, oxygen, et cetera spectra can be determined by calibration, wherein these spectra can be stored in a storing unit of the spectrometer 39. After a spectrum has been generated for an actual measurement, this spectrum can be compared with the spectra stored in the storing unit for determining the material actually illuminated by the light. In particular, if it is assumed that different tissue types consist of different material combinations, the spectrometer 39 can be adapted to determine the respective tissue type by investigating the spectrum. For example, if it is known that a certain tissue type consists of a certain material combination, by spectrally analyzing the actually measured spectrum it can be determined which material combination is actually illuminated and, thus, which tissue type is actually illuminated. The imaging apparatus can therefore be adapted to allow determining which tissue type is in front of a catheter tip or a needle tip.

The catheter control unit 10 further comprises a registration unit 13 for registering at least one of the first ultrasound image and the second ultrasound image with the fluoroscopy image. For registering two images the registration unit 13 preferentially uses an element being visible in the two images. This element is, for example, an ultrasound sensor, a biopsy needle, an energy application elements like an electrode, et cetera. The registered images are preferentially overlaid by an overlay unit 14 for generating an overlay image. The overlay unit 14 is preferentially adapted to overlay the fluoroscopy image with at least one of the ultrasound images.

The catheter control unit 10 further comprises a navigation unit 24 for allowing the catheter 6, in particular, the distal end 29 of the catheter 6, to be navigated to a desired location within the object 2 depending at least on the first ultrasound image and the second ultrasound image. In this embodiment, the navigation unit 24 is adapted to allow the catheter to be navigated also depending on the further images, i.e. the fluoroscopy image, the spectral image and preferentially also the third ultrasound image.

The navigation unit 24 can be adapted to allow a user to navigate the catheter 6 completely by hand or semi-automatically depending on at least the first ultrasound image and the second ultrasound image. The navigation unit 14 can also be adapted to navigate the catheter 6 automatically depending on at least the first ultrasound image and the second ultrasound image.

The catheter 6 preferentially comprises build-in guiding means (not shown in FIG. 1), which can be controlled by the navigation unit 24. The catheter 6 can, for example, be steered and navigated by the use of steering wires in order to guide the distal end 29 of the catheter 6 to the desired location within the object 2.

The first ultrasound image having a smaller spatial resolution can be used to coarsely navigate the housing to the desired location, and the second ultrasound image having a finer spatial resolution can be used to navigate the housing more precisely to the desired location. Moreover, the first ultrasound image can be used to image a first region of interest located at a larger depth within the object 2 and the second ultrasound image can be used to image a second region of interest being located at a smaller depth within the object 2. The first ultrasound sensor 4 and the second ultrasound sensor 5 can be operated simultaneously, in order to provide feedback for coarse guidance and simultaneously high resolution information, in particular, from the vicinity of the distal end 29 of the catheter 6 which houses, for example, a tip of a biopsy needle. However, the first ultrasound sensor and the second ultrasound sensor can also be operated sequentially, in order to firstly provide the coarse information and subsequently provide the fine information. Also further ultrasound images like the third ultrasound image having different spatial resolutions and different penetration depths can be used for navigation purposes. Also the third ultrasound sensor can be operated simultaneously with the first and second ultrasound sensors, or the three ultrasound sensors can be operated sequentially.

In a preferred embodiment, the fluoroscopy image or, if, for example, a computed tomography device or a magnetic resonance device is used as a second image generation device, a computed tomography image or a magnetic resonance image, is used for very coarse information on approaching a desired location within the object. Then, the registered first ultrasound image is overlaid over the fluoroscopy, computed tomography or magnetic resonance image for providing coarse long range information, wherein, since the spatial resolution of the first ultrasound image is assumed to be larger than the spatial resolution of the fluoroscopy, computed tomography or magnetic resonance image, the accuracy of approaching the desired location is improved. If the desired location has coarsely been reached, a fine alignment of the catheter tip is preferentially performed based on the registered second ultrasound image overlaid over the fluoroscopy, computed tomography or magnetic resonance image. After finally being aligned, for example, a biopsy needle can be inserted into the object. The second ultrasound image can also be used to provide high resolution information on lesion/tumor boundary for resection, wherein during the resection the first ultrasound image can be used for monitoring the resection within, for example, the tissue of the object. The second ultrasound image can, for example, be used for ensuring that neighbor tissue, which should not be resected, is not damaged by the biopsy needle.

The imaging apparatus can be adapted to perform the navigation procedure automatically by using a corresponding roboter unit. A feedback loop using the different images having different spatial resolutions and optionally also using the spectral information received from the spectrometer can be used for adjusting a navigation trajectory, along which an interventional device should be moved. An adjustment of the navigation trajectory may, for example, be necessary if critical structures like blood vessels or nerves are located in the vicinity of the navigation trajectory. Also, for example, an insertion of a needle as the interventional device can be automatically performed by the roboter unit.

If a further ultrasound sensor like the third ultrasound sensor 3 is used, which generates an ultrasound image having a spatial resolution being different to the spatial resolutions of the first and second ultrasound images, this further ultrasound image can also be used for navigating the catheter tip to the desired location.

Adjacent frequencies are preferentially separated by at least the sum of half the bandwidth of the respective ultrasound sensors. If, for example, only the first ultrasound sensor 4 and the second ultrasound sensor 5 are used for imaging, the first frequency is preferentially in the range of 1 to 10 MHz and the second frequency is preferentially in the range of 20 to 40 MHz.

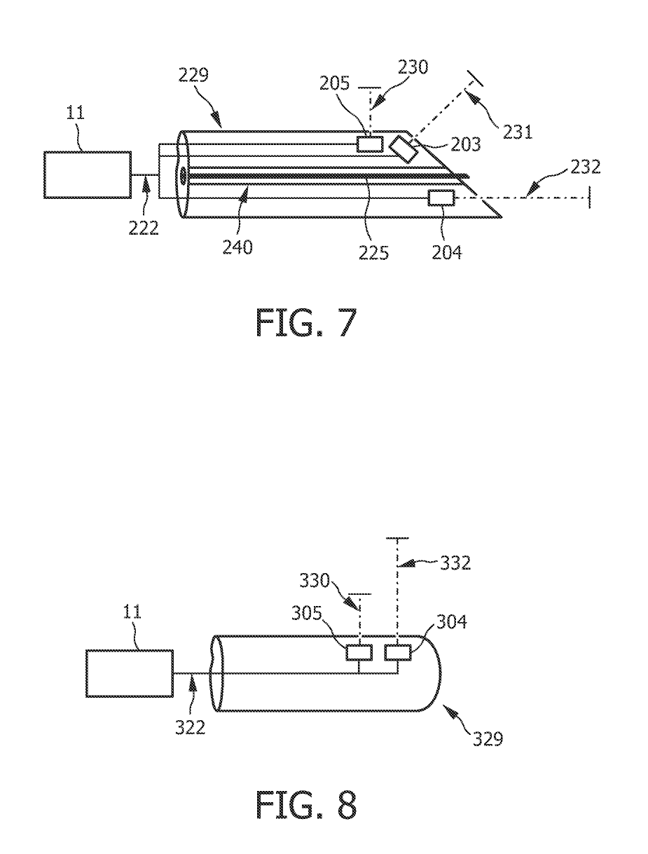

FIG. 7 shows schematically and exemplarily a further embodiment of a distal end 229 of the catheter, which, for example, can be used instead of the distal end 29 of the catheter shown in FIG. 2. The distal end 229 of the catheter comprises three ultrasound sensors. A first ultrasound sensor 204 sensing at a first frequency, a second ultrasound sensor 205 sensing at a second frequency and a third ultrasound sensor 203 sensing at a third frequency. Also in this embodiment the first frequency is smaller than the third frequency and the second frequency is larger than the third frequency. Because of the different frequencies, the three ultrasound sensors 203, 204, 205 have different imaging ranges 230, 231, 232, different depths of penetrating into, for example, tissue of the object, and different spatial resolutions. The different ultrasound sensors 203, 204, 205 are arranged to sense in different directions. In particular, the first ultrasound sensor is arranged to sense in a longitudinal direction with respect to the catheter, the second ultrasound sensor 205 is arranged to sense in a transverse direction with respect to the catheter, and the third ultrasound sensor 203 is arranged to sense in an oblique direction between the longitudinal direction and the transverse direction. The catheter further comprises a biopsy channel 240 through which a biopsy needle 225 can be forwarded for performing a biopsy operation. The biopsy needle 225 can be controlled by a biopsy control unit 26 comprised by the catheter control unit 10. Also the ultrasound sensors 203, 204, 205 can be connected to the single driving unit 11 via a single wire 222.

FIG. 8 shows a further embodiment of a distal end 329 of a catheter. The distal end 329 of the catheter comprises two ultrasound sensors, a first ultrasound sensor 304 and a second ultrasound sensor 305, which are arranged to sense in a transverse, side-looking direction. The first ultrasound sensor senses at a first frequency being smaller than a second frequency at which the second ultrasound sensor senses the interior of the object. The ultrasound sensors 304, 305 comprise therefore different imaging ranges 330, 332, which allow the ultrasound sensors 304, 305 to generate ultrasound images at different depths within the object. The first and second ultrasound sensors 304, 305 are connected to the driving unit 11 via a single wire 322.

FIG. 9 shows a further embodiment of a distal end of a catheter. The distal end 429 of the catheter shown in FIG. 9 comprises four ultrasound sensors 404, 405, 441, 442, which are arranged to sense the interior of the object in a transverse direction with respect to the catheter. A first ultrasound sensor 404 and a third ultrasound sensor 441 are directed in opposite directions and also a second ultrasound sensor 405 and a fourth ultrasound sensor 442 are directed in opposite directions. The first ultrasound sensor operates at a first frequency being smaller than a second frequency at which the second ultrasound sensor 405 is operated. The third ultrasound sensor 441 is preferentially operated at a third frequency being equal to the first frequency, and the fourth ultrasound sensor 442 is preferentially operated at a fourth frequency being equal to the second frequency. Since the first and second ultrasound sensors 404, 405 are operated at different frequencies, their imaging ranges 430, 432 are different. This allows the first and second ultrasound sensors 404, 405 to image the interior of the object at different depths. Moreover, an ultrasound image generated from first ultrasound sensing signals of the first ultrasound sensor 404 has a smaller spatial resolution than an ultrasound image generated from second ultrasound sensing signals from the second ultrasound sensor 405. Thus, ultrasound images having different spatial resolutions can be determined at different depths. Since also the third and fourth ultrasound sensors 441, 442 are operated at different frequencies, also these ultrasound sensors can be used for sensing the interior of the object at different depths with different spatial resolutions. The corresponding imaging ranges are indicated in FIG. 9 by reference numbers 443, 444. The catheter further comprises a biopsy channel 440 in which a biopsy needle 425 can be forwarded for performing a biopsy operation. Also the biopsy needle 425 can be connected to the biopsy control unit 26 for performing the biopsy operation. The ultrasound sensors 404, 405, 441, 442 are connected to the single driving unit 11 via a single wire 422.

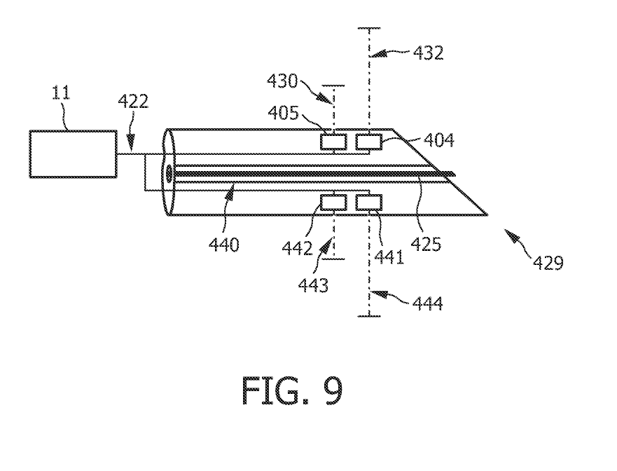

FIG. 10 illustrates schematically and exemplarily the navigation of the distal end 229 of the catheter 6 to target tissue 45 being, for example, a lesion. The distal end 229 of the catheter is described above in more detail with reference to FIG. 7. In this example, the three ultrasound sensors 203, 204, 205 sequentially or simultaneously sense different regions 46, 47, 48 of the person 27. The corresponding ultrasound images are used together with the fluoroscopy image generated by the second image generation device 7 for navigating the distal end 229 of the catheter 6 to the target tissue 45. Since three adjacent regions 46, 47, 48 are imaged with ultrasound, the probability that the target tissue 45 is not found is very low. The ultrasound images allow to monitor the spatial location of the distal end 229 in relation to the target tissue location 45 and to use this relative spatial location to navigate the distal end 229 to the target tissue 45.

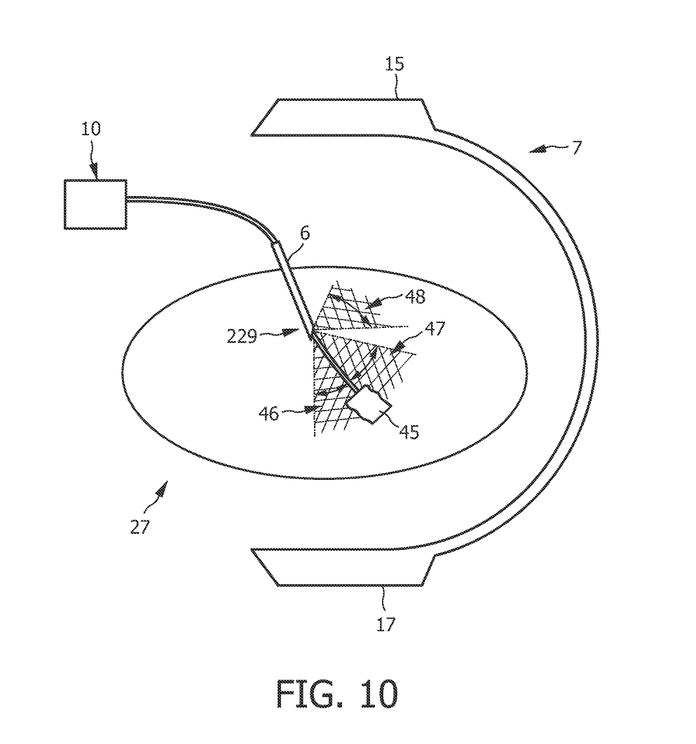

In the following, an embodiment of an imaging method for imaging an interior of an object 2 will exemplarily be described with reference to a flowchart shown in FIG. 11.

In step 501, the interior of the object is sensed at a first frequency by a first ultrasound sensor, wherein first ultrasound sensing signals are generated being indicative of the interior of the object. In step 502, the interior of the object is sensed at a second frequency by a second ultrasound sensor, wherein second ultrasound sensing signals are generated being indicative of the interior of the object. The first frequency is smaller than the second frequency such that the first ultrasound sensor senses the interior of the object with smaller spatial resolution and the second ultrasound sensor senses the interior of the object with larger spatial resolution.

In step 503, a first ultrasound image having a smaller spatial resolution is generated from the first ultrasound sensing signals and a second ultrasound image having a larger spatial resolution is generated from the second ultrasound sensing signals by an ultrasound image generation unit.

Steps 501 and 502 can be performed simultaneously or sequentially, wherein if steps 501 and 502 are performed sequentially, after first or second ultrasound sensing signals, respectively, have been generated, the first ultrasound image or the second ultrasound image, respectively, is generated in step 503, without waiting for the other ultrasound sensing signals, i.e. the second or first ultrasound sensing signals, respectively. For example, firstly steps 501 and 503 can be performed in a loop, wherein several first ultrasound images are generated, without generating a second ultrasound image, for example, for coarse navigation purposes, wherein then steps 502 and 503 can be performed one time or in a loop for generating one or several second ultrasound images, without generating a first ultrasound image, for example, for performing a more precise navigation of a catheter.

If the catheter comprises an influencing element like a biopsy needle or an energy application element, for example, as described above with reference to FIG. 3, 7 or 9, this catheter together with the imaging apparatus can be regarded as an influencing apparatus for influencing an interior of an object. This influencing apparatus preferentially comprises the influencing element being, for example, a biopsy needle or an energy application element, for influencing the object, an imaging apparatus comprising at least the first ultrasound sensor, the second ultrasound sensor, the ultrasound image generation unit, and the catheter, and the navigation unit for navigating the influencing element to a desired location within the interior of the object depending on at least the first ultrasound image and the second ultrasound image.

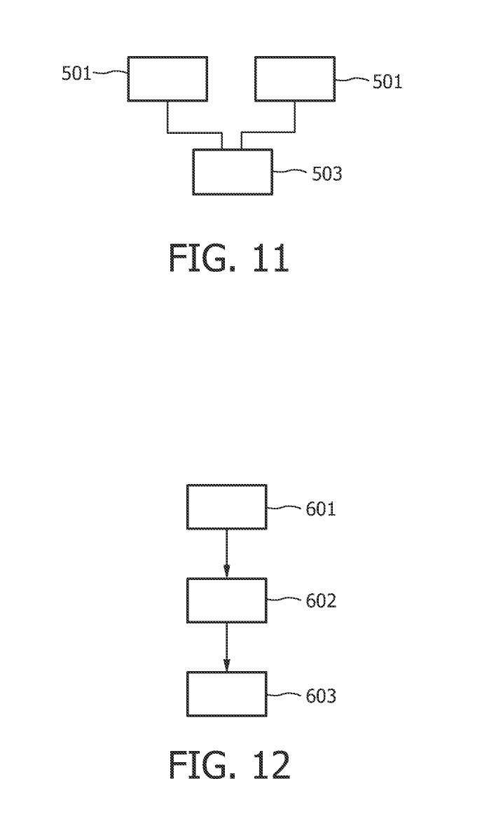

A corresponding influencing method for influencing an interior of an object will in the following exemplarily be described with reference to a flowchart shown in FIG. 12.

In step 601, a first ultrasound image and a second ultrasound image are generated by the imaging apparatus as described above with reference to FIG. 11, and in step 602, the influencing element 225 is navigated to the desired location within the interior of the object depending on at least the first ultrasound image and the second ultrasound image by the navigation unit. Steps 601 and 602 can be performed in a loop such that the navigation of the influencing element can dynamically be adjusted to the actually generated first and/or second ultrasound images, in particular, in realtime. Also further images like an image generated by the second image generation device, which may be overlaid over the first ultrasound image and/or the second ultrasound image, and/or spectra or determined certain kinds of material, in particular, certain kinds of tissue, determined by the spectrometer can be used for navigating the influencing element to the desired location.

In an embodiment, firstly the first ultrasound image is generated and a coarse navigation is performed based on this first ultrasound image. The generation of the first ultrasound image and the navigation based on the first ultrasound image can be performed in a loop such that the navigation of the influencing element can dynamically be adjusted to the actually generated first ultrasound image. After the influencing element has coarsely been navigated to the desired location, the second ultrasound image can be generated and a more precise fine navigation procedure can be performed based on the second ultrasound image. Also the generation of the second ultrasound image and the navigation of the influencing element based on the second ultrasound image can be performed in a loop such that also the finer navigation of the influencing element can dynamically be adjusted to the actually generated second ultrasound image, in particular, in realtime.

If the influencing element has reached the desired location, the influencing element influences the object at the desired location in step 603. For example, a biopsy operation is performed or energy is applied at the desired location.

Interactive realtime monitoring of a biopsy needle tip spatial location in relation to a desired location being preferentially a lesion location is a long lasting clinical requirement. Current ultrasound guided needle tracking is generally not satisfactory in deep seated lesions, neither it is used in superficial lesions where bones and air are the obstructing factors. Other imaging modalities are less applicable due to its non-realtime response (note that X-ray fluoroscopy imaging does have realtime scanning feedback, but no satisfactory contrast resolution). The imaging apparatus can provide an imaging solution which consists of scanning ultrasound sensors attached to a biopsy needle tip that actively scan tissue surrounding while being penetrated into a human body.

Accurate needle-mediated tissue sampling is a problem, especially in small and deep seated lesions. Following problems can occur while performing needle-mediated biopsies/drainages.

Image guided needle navigation based on computed tomography or magnetic resonance pre-acquired scans results in a rather high number of misplaced needles, mainly because of not having interactive assessment of organ/tissue motion. Ultrasound guided needle navigation is considered to be the gold standard for superficial lesions in solid anatomy. However, limited ultrasound penetration and signal interference with bony anatomy and air makes this technique not usable in deep seated lesions, lungs, anatomy around bowels, et cetera. X-ray fluoroscopy provides rather satisfactory guidance in bone biopsies and some pain related spinal procedures, but is not used in a large variety of biopsies and drainages because of a narrow dynamic range. Photonic needles provide a very good solution for interactive detection of lesion boundaries and lesion content, but due to their limited penetration rate they may have accuracy problems while detecting a lesion location.

The imaging apparatus and influencing apparatus can be used to interactively detect a lesion location and boundaries in respect to a current needle location while penetrating tissue, to interactively detect lesion motion that helps defining a most optimal needle insertion course and helps in steering the needle towards the target, to interactively check the final needle tip placement inside the lesion mass, and/or to interactively monitor lesion modifications while injecting contrast or therapeutic agents, performing ablation, et cetera.

Although in the above described embodiments preferentially the imaging apparatus is combined with an influencing element being a biopsy needle or an energy application element like an ablation electrode, the imaging apparatus can also be combined with other elements, in particular, other influencing elements, which can be integrated within the catheter tip, for example, a contrast injection element can be located within the catheter, wherein the contrast injection element can be navigated to a desired location by the navigation unit, which uses preferentially at least the first and second ultrasound images for injecting the contrast agent. For the coarse navigation the first ultrasound sensor has preferentially a first frequency in the range of 1 to 9 or 1 to 10 MHz for which the penetration depth is in the range of 10 cm or more, if the catheter is introduced into a person. For the fine navigation the second ultrasound sensor has preferentially a second frequency in the range of 20 to 40 MHz with a penetration depth up to about 2 to 3 cm, if the catheter is introduced into a person.

The ultrasound sensors might be single element transducers or ultrasound transducer arrays, giving larger coverage of the surrounding region, in particular, of the surrounding tissue.

If an embodiment is used comprising a needle like a biopsy needle, the fluoroscopy image being preferentially an X-ray image can be combined with contrast of tissue coming from the first and/or second ultrasound image by overlaying registered fluoroscopy and ultrasound images. This can improve localization of, for example, lesions, cancer seeds, abnormalities in tissue, et cetera, and may correct for slight position changes in realtime, when the needle is advanced towards the desired location.

In minimally-invasive treatments, for example, oncology, cardiac arrhythmias and valve repairs, thin devices are used such as needles and catheters. The importance of the size of the treatment and monitoring device is crucial, because it is usually limited by its pathway like blood vessels and related directly to the post operatory trauma Thinner catheters are preferred for example in the treatment of atrial fibrillation due to the fact that the catheter has to pass femoral vein and the septum that separates the two atria. Most of the future minimally-invasive devices will probably have to combine diagnosis, navigation and treatment possibilities. The diameter restrictions of the devices lead to smart combinations of these functionalities. The above described embodiments, which use the same driving unit, i.e. the same driving electronics, for different ultrasound sensors reduce the leads that have to be fed through the minimally-invasive device allowing for smaller diameters, but also incorporates cost effective driving electronics by reducing the required number of hardware components around the device. Moreover, the mechanical construction of minimally-invasive inventional devices can be simplified.

The imaging apparatus can comprise a display for displaying the different images and optionally the spectral information for allowing a user to navigate depending on the different images and the optionally provided spectral information. The imaging apparatus can also be adapted to allow a person to select which ultrasound sensor should be operated and which image should be shown on the display. In particular, the imaging apparatus can be adapted to provide a zooming function. For example, referring again to FIG. 2, firstly the first ultrasound image generated by the first ultrasound sensor 4 and having the smallest spatial resolution can be shown on the display. Then, if a user wants to zoom in, the user can select the second ultrasound image generated by the second ultrasound sensor 5 or the third ultrasound image generated by the third ultrasound sensor 3. In general, the imaging apparatus can be adapted to allow a user to switch between the different ultrasound images having different spatial resolutions.

In an embodiment, only a first ultrasound sensor and a second ultrasound sensor are present, wherein the first ultrasound sensor comprises a first frequency of 5 MHz and the second ultrasound sensor comprises a second frequency of 30 MHz. The bandwidth of the first ultrasound sensor and the second ultrasound sensor is preferentially about 40%.

The imaging apparatus and the influencing apparatus can be adapted to be used for intravascular imaging and treatment, in particular, for performing a biopsy in oncology. In particular, the imaging apparatus is preferentially adapted to be used as a cardiac treatment catheter, an interventional device for intravascular purposes, an interventional device for oncology, et cetera. Preferentially, the imaging apparatus and the influencing apparatus are adapted to be used in interactive realtime monitoring of an interventional device spatial position in relation to a lesion location. However, the imaging apparatus can also be adapted for investigation purposes only, without providing a treatment option, wherein tissue can be investigated at various depths with different spatial resolutions.

It should be noted that the distal ends of the catheters described above with reference to FIGS. 2, 3 and 7 to 9 can comprise more elements than shown in the figures. Moreover, elements shown in different figures can be combined to a distal end of a catheter comprising these elements shown in different figures. For example, also the distal ends of the catheter shown in FIGS. 2 and 8 can comprise a biopsy channel with a biopsy needle, and also the arrangement and number of ultrasound sensors shown in FIGS. 2, 3 and 8 can be provided in the distal ends of the catheter shown in FIGS. 7 and 9. Moreover, the optical fibers shown in FIG. 2 can be provided also in the other catheters shown in the figures.

The different possible catheters, in particular, the different possible distal ends of the catheters, can be used together with the other elements shown in FIG. 1 for generating at least the ultrasound images and preferentially also for navigating the respective distal end of the catheter to the desired location.

Although in the above described embodiments certain numbers of ultrasound sensors have been described, the imaging apparatus can also comprise another number of ultrasound sensors equal to or larger than two.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Functions like the registration and overlaying performed by one or several units or devices can be performed by any other number of units or devices. The control of the imaging apparatus in accordance with the above described imaging method and/or the control of the influencing apparatus in accordance with the above described influencing method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to an imaging apparatus for imaging an interior of an object. The imaging apparatus comprises a first ultrasound sensor and a second ultrasound sensor for sensing the interior of the object at different frequencies, wherein the ultrasound sensing signals from the first ultrasound sensor are used for generating a first ultrasound image and a the ultrasound sensing signals from the second ultrasound sensor are used for generating a second ultrasound image. A larger frequency generally provides a smaller depth of penetrating the interior of the object and a larger spatial resolution than a smaller frequency. The imaging apparatus can therefore provide the capability of simultaneously imaging the interior of the object with different spatial resolutions and at different penetration depths. This allows the imaging apparatus to improve the quality of imaging the interior of the object.

1. An imaging apparatus for imaging an interior of an object (2), the imaging apparatus (1) comprising:
a first image generation device including:
a) a first ultrasound sensor (4) for sensing the interior of the object (2) at a first frequency, wherein first ultrasound sensing signals are generated being indicative of the interior of the object,
b) a second ultrasound sensor (5) for sensing the interior of the object (2) at a second frequency, wherein second ultrasound sensing signals are generated being indicative of the interior of the object (2), wherein the first frequency is smaller than the second frequency such that the first ultrasound sensor (4) is adapted to sense the interior of the object with smaller spatial resolution and the second ultrasound sensor (5) is adapted to sense the interior of the object with larger spatial resolution,
c) an ultrasound image generation unit (12) for generating a first ultrasound image having a smaller spatial resolution from the first ultrasound sensing signals and a second ultrasound image having a larger spatial resolution from the second ultrasound sensing signals,
a housing (6) for housing at least the first ultrasound sensor and the second ultrasound sensor, the housing (6) being adapted to be introducible into the object.

2. The imaging apparatus as defined in claim 1, wherein the imaging apparatus further comprises:
a second image generation device (7) for generating a third image of the object,
an overlay unit (14) for overlaying the third image with at least one of the first ultrasound image and the second ultrasound image.

3. The imaging apparatus as defined in claim 2, wherein the second image generation device (7) comprises:
a) a radiation source (15) for generating radiation (16) for traversing the object (2),
b) a detector (17) for generating detection values depending on the radiation after having traversed the object (2),
c) an image reconstruction unit (19) for reconstructing the third image from the generated detection values.

4. The imaging apparatus as defined in claim 2, wherein the imaging apparatus (1) further comprises a registration unit (13) for registering at least one of the first ultrasound image and the second ultrasound image with the third image.

5. The imaging apparatus as defined in claim 1, wherein the imaging apparatus further comprises a driving unit (11) for driving the first ultrasound sensor and the second ultrasound sensor.

6. The imaging apparatus as defined in claim 5, wherein the driving unit (11) is connected to the first ultrasound sensor (4) and the second ultrasound sensor (5) via a single wire (22).

7. The imaging apparatus as defined in claim 5, wherein the driving unit (11) is adapted to receive combined ultrasound sensing signals (36) from the first ultrasound sensor (4) and the second ultrasound sensor (5) comprising the first ultrasound sensing signals (38) and the second ultrasound sensing signals (37), wherein the driving unit (11) comprises a filtering unit (23) for filtering the first ultrasound sensing signals (38) and the second ultrasound sensing signals (37) out of the combined ultrasound sensing signals (36).

8. The imaging apparatus as defined in claim 1, wherein the imaging apparatus further comprises a navigation unit (24) for allowing the housing (6) to be navigated to a desired location within the object (2) depending on at least the first ultrasound image and the second ultrasound image.

9. The imaging apparatus as defined in claim 1, wherein the first frequency and the second frequency are separated by at least the sum of half the bandwidth of the first ultrasound sensor and the second ultrasound sensor.

10. The imaging apparatus as defined in claim 1, wherein the first frequency is in the range of 1 to 10 MHz and the second frequency is in the range of 20 to 40 MHz.

11. An influencing apparatus for influencing an interior of an object, the influencing apparatus comprising:
an influencing element (225) for influencing the object (2),
an imaging apparatus for generating a first ultrasound image and a second ultrasound image as defined in claim 1,
a navigation unit (24) for navigating the influencing element to a desired location within the interior of the object depending on at least the first ultrasound image and the second ultrasound image.

12. An imaging method for imaging an interior of an object (2), the imaging method comprising:
a) sensing the interior of the object (2) at a first frequency by a first ultrasound sensor (4), wherein first ultrasound sensing signals are generated being indicative of the interior of the object,
b) sensing the interior of the object (2) at a second frequency by a second ultrasound sensor (5), wherein second ultrasound sensing signals are generated being indicative of the interior of the object (2), wherein the first frequency is smaller than the second frequency such that the first ultrasound sensor (4) senses the interior of the object with smaller spatial resolution and the second ultrasound sensor senses the interior of the object with larger spatial resolution,
c) generating a first ultrasound image having a smaller spatial resolution from the first ultrasound sensing signals and a second ultrasound image having a larger spatial resolution from the second ultrasound sensing signals by an ultrasound image generation unit (12), wherein at least the first ultrasound sensor and the second ultrasound sensor are housed within a housing (6) which is adapted to be introducible into the object (2).

13. An influencing method for influencing an interior of an object, the influencing method comprising:
generating a first ultrasound image and a second ultrasound image by an imaging apparatus as defined in claim 12,
navigating an influencing element (225) for influencing the object (2) to a desired location within the interior of the object depending on at least the first ultrasound image and the second ultrasound image by a navigation unit (24), and
influencing the object (2) with the influencing element (225).

14. An imaging computer program for imaging an interior of an object (2), the imaging computer program comprising program code means for causing a computer to carry out the steps of the imaging method as defined in claim 12.

15. An influencing computer program for influencing an interior of an object (2), the influencing computer program comprising program code means for causing a computer to carry out the steps of the influencing method as defined in claim 13.
